(19) United States
(12) Patent Application Publication
(10) Pub. No.: US 2022/0304884 A1
(43) Pub. Date: Sep. 29, 2022

(54) FOOT MASSAGE ASSEMBLY FOR VEHICLE

(71) Applicants: Hyundai Motor Company, Seoul (KR); Kia Corporation, Seoul (KR); Hyundai Transys Inc, Seosan-si (KR)

(72) Inventors: Byeong-Seon SON, Seoul (KR); Tae-Hoon Lee, Suwon-Si (KR); Ji-Hwan Kim, Seoul (KR); Seon-Chae Na, Yongin-Si (KR); Sang-Ho Kim, Incheon (KR); Sang-Hoon Park, Incheon (KR); Seong-Yun Yeo, Seongnam-Si (KR); Sung-Hoon Kim, Seoul (KR)

(73) Assignees: Hyundai Motor Company, Seoul (KR); Kia Corporation, Seoul (KR); Hyundai Transys Inc, Seosan-si (KR)

(21) Appl. No.: 17/532,668

(22) Filed: Nov. 22, 2021

(30) Foreign Application Priority Data
Mar. 25, 2021 (KR) 10-2021-0039083

(51) Int. Cl. A61H 9/00; B60N 2/90; B60N 2/56

(52) U.S. Cl. CPC A61H 9/0078; B60N 2/976; B60N 2/5685; A61H 2201/0149; A61H 2201/0207; A61H 2201/0228; A61H 2201/1238; A61H 2201/1409; A61H 2201/1654; A61H 2205/12

(57) ABSTRACT

A foot massage assembly for a vehicle, may simultaneously provide a foot massage and a heating function and may include a plate mounted on a seatback for a vehicle, a bladder unit spaced from the plate at an interval and provided with a plurality of bladder cells, wherein air is supplied to or exhausted from an inside of the bladder cells for pressurizing passenger's soles, and a heat wire mat positioned to cover the bladder unit and provided with a heat wire.

### CROSS-REFERENCE TO RELATED APPLICATION

The present application claims priority to Korean Patent Application No. 10-2021-0039083, filed on Mar. 25, 2021, the entire contents of which is incorporated herein for all purposes by this reference.

### BACKGROUND OF THE INVENTION

### Field of the Invention

The present invention relates to a foot massage assembly for a vehicle mounted on a seatback for a vehicle to massage feet of a passenger in a rear seat, and to a foot massage assembly for a vehicle which simultaneously provides a foot massage and a heating function.

### Description of Related Art

Various convenience devices for a passenger's convenience are mounted inside a vehicle.

As one of such convenience devices, there is a foot massage assembly for massaging feet of a passenger in a rear seat mounted on a seat of the vehicle.

As illustrated in FIG. 1, there is a foot massage assembly 120 mounted on a seatback 111 of a seat 110 for the vehicle to operate in conjunction therewith when a foot rest 112 is deployed. A passenger on a rear seat deploys the foot rest 112, and contacts his or her foot with the foot massage assembly 120 to massage the foot.

As illustrated in FIG. 2, the foot massage assembly 120 includes a plate 121, a plurality of bladder cells 122 repeating expansion and restoration by a pneumatic pressure, and an air hose 123 connecting an air pump 126 and the bladder cell 122.

The foot massage assembly 120 according to the related art repeats a process in which the bladder cell 122 is expanded and restored to massage a passenger's sole.

However, the foot massage assembly 120 according to the related art massages the passenger's soles simply, and does not provide a function of heating the feet during massage. Recently, a high-end specification is in progress, and the foot massage assembly 120 is applied to a luxury vehicle, but merchantability is not more enhanced because the feet are not heated during massage.

The information disclosed in this Background of the Invention section is only for enhancement of understanding of the general background of the invention and may not be taken as an acknowledgement or any form of suggestion that this information forms the prior art already known to a person skilled in the art.

### BRIEF SUMMARY

Various aspects of the present invention are directed to providing a foot massage apparatus for a vehicle, which provides a heating function to heat his or her feet while acupressure is applied to passenger's soles in a rear seat.

A foot massage apparatus for a vehicle according to various exemplary embodiments of the present invention for achieving the object includes a plate mounted on a seatback for a vehicle, a bladder unit spaced from the plate at an interval and provided with a plurality of bladder cells, wherein air is supplied to or exhausted from an inside of the bladder cells for pressurizing passenger's soles, and a heat wire mat positioned to cover the bladder unit and provided with a heat wire.

The heat wire is configured for being uniformly distributed and provided on the heat wire mat.

The heat wire is configured for being provided in a region in which the bladder cells are not provided.

The heat wire is configured for being provided in a region in which an air hose for supplying pressurized air to the bladder cells is not provided.

The heat wire is configured for being provided to overlap an air hose for supplying pressurized air to the plurality of bladder cells.

The heat wire is configured for being arranged to be tilted with respect to a vertical direction or a horizontal direction of the heat wire mat.

The heat wire is configured for being provided on the heat wire mat so that left and right sides of the heat wire are linearly symmetric to each other.

The foot massage apparatus for the vehicle is configured for further including an air pump for pressurizing air to supply the pressurized air, and a control valve provided between the air pump and an air hose for selectively supplying pressurized air to the bladder cells to selectively interrupt flow of the air.

The foot massage apparatus for the vehicle is configured for further including a control unit of operating the air pump for supplying the pressurized air to the bladder cell and connecting a power source to the heat wire.

The bladder unit is configured for being provided with a felt coupled to the plate, and the bladder cell is configured for being attached to the felt at an interval.

The bladder cell is configured for being attached to the felt by thermal fusing.

A sponge for buffering is configured for being provided between the bladder unit and the heat wire mat.

A through hole is configured for being formed in a portion in which the bladder cell is disposed in the sponge.

The foot massage apparatus for the vehicle is configured for further including a cover for covering the heat wire mat.

The foot massage apparatus is configured for being provided to be exposed when a foot rest hinged to the seatback is deployed.

Furthermore, a foot massage apparatus for a vehicle is configured for including a plate mounted on a seatback for a vehicle, a bladder unit spaced from the plate at an interval and provided with a plurality of bladder cells, wherein air is supplied to or exhausted from an inside of the bladder cells for pressurizing passenger's soles, and a heat wire mat having a heat wire arranged in a region in which the plurality of bladder cells is not provided and positioned to cover the bladder unit.

The heat wire is configured for being uniformly distributed and provided in a region other than the plurality of bladder cells.

The heat wire is configured for being provided to bypass an air hose fluidically connected to a bladder cell among the plurality of bladder cells, and to overlap the air hose connected to other bladder cells disposed above and below the bladder cell among the plurality of bladder cells.

The heat wire is configured for overlapping the air hose to be perpendicular to a direction in which the air hose is arranged upon overlapping the air hose.

The foot massage apparatus for the vehicle according to various exemplary embodiments of the present invention having the above configuration may heat the feet while massaging the passenger's feet, thereby reducing the passenger's fatigue and maximizing comfort.

Furthermore, when the heat wire is provided not to overlap the bladder cell, it is possible to prevent the heat wire from being damaged by the operation of the bladder cell, thereby improving durability.

Furthermore, it is possible to improve the merchantability of the vehicle by satisfying consumer's advanced demands.

The methods and apparatuses of the present invention have other features and advantages which will be apparent from or are set forth in more detail in the accompanying drawings, which are incorporated herein, and the following Detailed Description, which together serve to explain certain principles of the present invention.

### BRIEF DESCRIPTION OF THE DRAWINGS

FIG. 1 is a perspective diagram illustrating a state where a foot massage assembly for a vehicle according to the related art is mounted on a seat.

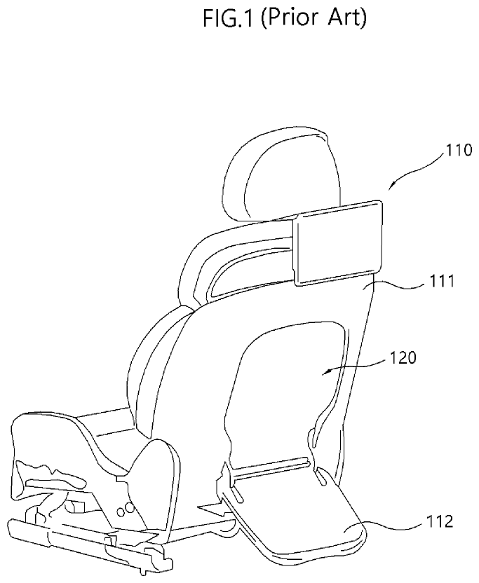

FIG. 2 is a perspective diagram illustrating a structure of the foot massage assembly for the vehicle according to the related art.

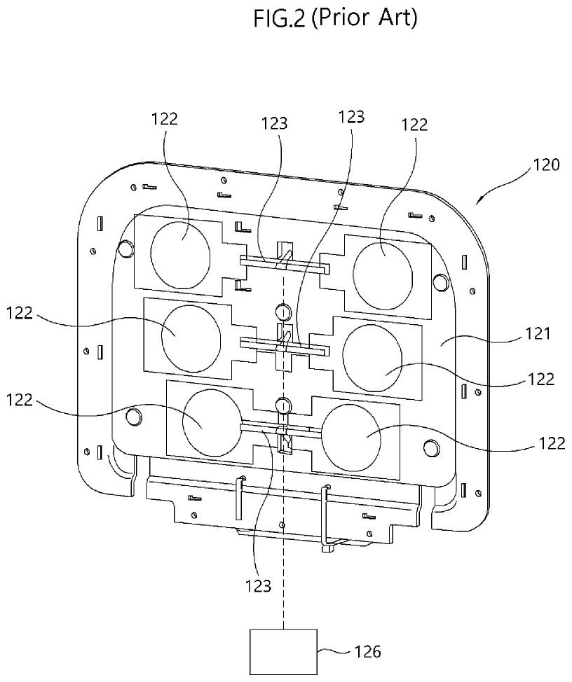

FIG. 3 is a plan diagram illustrating a foot massage assembly for a vehicle according to various exemplary embodiments of the present invention.

FIG. 4 is a schematic diagram illustrating a use state of the foot massage assembly for the vehicle according to the exemplary embodiment of the present invention.

FIG. 5 is an exploded perspective diagram illustrating the foot massage assembly for the vehicle according to the exemplary embodiment of the present invention.

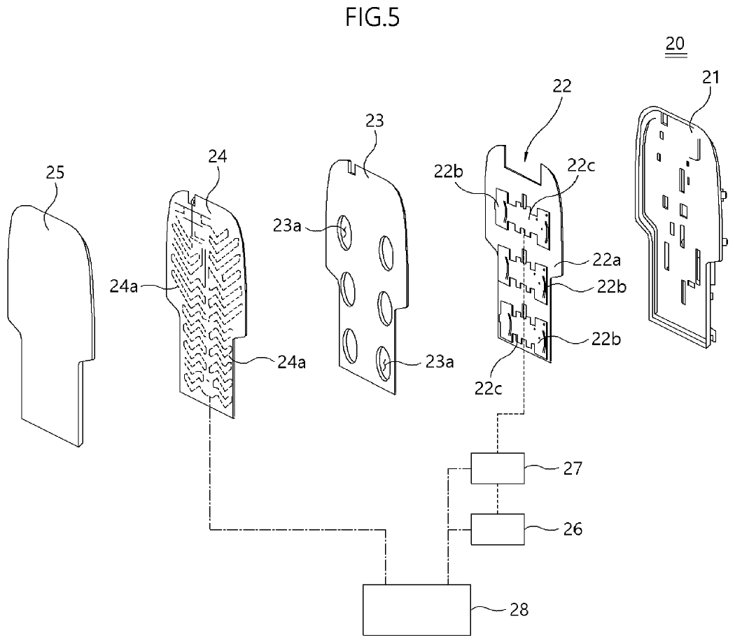

FIG. 6 is a plan diagram illustrating a foot massage assembly for a vehicle according to various exemplary embodiments of the present invention.

FIG. 7 is a plan diagram illustrating the foot massage assembly for the vehicle according to various exemplary embodiments of the present invention.

It may be understood that the appended drawings are not necessarily to scale, presenting a somewhat simplified representation of various features illustrative of the basic principles of the present invention. The specific design features of the present invention as disclosed herein, including, for example, specific dimensions, orientations, locations, and shapes will be determined in part by the particularly intended application and use environment.

In the figures, reference numbers refer to the same or equivalent parts of the present invention throughout the several figures of the drawing.

### DETAILED DESCRIPTION

Reference will now be made in detail to various embodiments of the present invention(s), examples of which are illustrated in the accompanying drawings and described below. While the present invention(s) will be described in conjunction with exemplary embodiments of the present invention, it will be understood that the present description is not intended to limit the present invention(s) to those exemplary embodiments. On the other hand, the present invention(s) is/are intended to cover not only the exemplary embodiments of the present invention, but also various alternatives, modifications, equivalents and other embodiments, which may be included within the spirit and scope of the present invention as defined by the appended claims.

Hereinafter, a foot massage assembly for a vehicle according to various exemplary embodiments of the present invention will be described in detail with reference to the accompanying drawings.

A foot massage assembly 20 for a vehicle according to various exemplary embodiments of the present invention includes a plate 21 mounted on a seatback 11 of the vehicle, a bladder unit 22 provided with a plurality of bladder cells 22b for pressurizing passenger's soles while air is supplied to or exhausted from the inside and disposed on the plate 21 to be spaced from each other, and a heat wire mat 24 provided with a heat wire 24a and positioned to cover the bladder unit 22.

The foot massage assembly 20 is mounted on the seatback 11 of the vehicle. Furthermore, the foot massage assembly 20 is not exposed by a foot rest 12 having a lower end portion hinged to the seatback 11 if not used, and is exposed to be usable by the passenger when the foot rest 12 is deployed.

The plate 21 allows the foot massage assembly 20 to maintain a constant shape. The plate 21 is formed to have constant shape and rigidity by injecting a synthetic resin or the like.

The plate 21 is fastened to the seatback 11 such that the foot massage assembly 20 is fastened to the seatback 11.

The bladder unit 22 includes a plurality of bladder cells 22b expanded and restored by air to pressurize the passenger's soles.

The bladder unit 22 includes a felt 22a formed in a certain area, the plurality of bladder cells 22b spaced from the felt 22a at an interval, and an air hose 22c for supplying air to the bladder cell 22b.

The felt 22a is formed in a shape corresponding to the shape of the plate 21. The felt 22a may be bonded to the plate 21 to fix the bladder unit 22 to the plate 21.

The bladder cell 22b pressurizes the passenger's soles while repeating the process of being expanded by filling air therein, or restored by exhausting the internal air.

A plurality of bladder cells 22b are spaced from the felt 22a at an interval, and attached to the felt 22a. The bladder cell 22b may also be directly provided on the plate 21, but preferably attached to the felt 22a to form the bladder unit 22, and the bladder unit 22 is attached to the plate 21.

As an example of a method for attaching the bladder cell 22b to the felt 22a, the bladder cell 22b is disposed on the felt 22a and then the bladder cell 22b and the felt 22a are thermally fused.

The air hose 22c becomes a passage through which the pressurized air is supplied to the bladder cell 22b, or the air is exhausted from the bladder cell 22b. The air hose 22c may be coupled to the bladder cell 22b after the bladder cell 22b is attached to the felt 22a.

An elastic member such as a sponge 23 covers the bladder unit 22. The sponge 23 covers and buffers the bladder unit 22, protecting the air hose 22c, and a portion connected to the air hose 22c and the bladder cell 22b in the bladder unit 22.

The sponge 23 is formed with a through hole 23a in the portion where the bladder cell 22b is positioned such that a force of pressurizing the feet by the expansion of the bladder cell 22b is directly delivered to the passenger's soles without being buffered by the sponge 23.

The heat wire mat 24 is provided to cover the sponge 23.

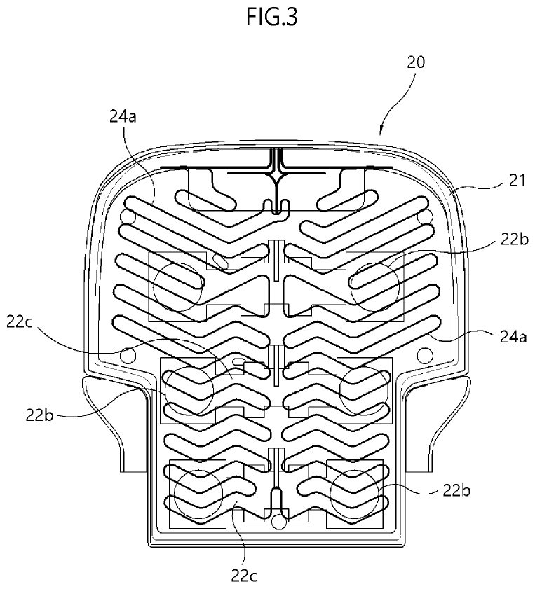

When a power source is supplied, the heat wire mat 24 is provided with the heat wire 24a heated. The heat wire 24a may be configured to be uniformly distributed in the entire region of the heat wire mat 24. Therefore, an amount of heat generated may be uniform in the heat wire mat 24. For example, as illustrated in FIG. 3, the heat wire 24a is provided such that a zig-zag pattern is repeated on the heat wire mat 24 such that the heat wire 24a is uniformly disposed in the heat wire mat 24. The heat wire 24a is arranged to be tilted with respect to a vertical direction or a horizontal direction of the heat wire mat 24, being arranged more uniformly. Furthermore, the heat wire 24a is provided such that left and right sides are linearly symmetric to each other on the heat wire mat 24. At the instant time, since the heat wires 24a are arranged to be tilted and the left and right sides are linearly symmetric to each other, the heat wire 24a may have a structure in which the left and right sides are symmetrically arranged in a form of being tilted in opposite directions.

Furthermore, the heat wires 24a may be arranged in a vortex form as well as the zig-zag shape on the heat wire mat 24.

As described above, when the heat wires 24a are uniformly arranged on the heat wire mat 24, there is an effect of evenly heating the foot massage assembly 20.

A cover 25 covers the heat wire mat 24 and directly contacts with the passenger's feet. For example, the cover 25 is made of a leather material and provided to cover the heat wire mat 24.

An air pump 26 pressurizes and discharges air such that the bladder cell 22b may be expanded.

A control valve 27 is connected to the air pump 26 and the air hose 22c to interrupt flow of the air. By operation of the control valve 27, the bladder cell 22b is expanded by sending the pressurized air discharged from the air pump 26 from the air hose 22c. Furthermore, the control valve 27 communicates with the outside such that the air inside the bladder cell 22b may be expelled to the outside to be restored to an original state. The bladder cell 22b repeats expansion and restoration by the control valve 27 to pressurize the passenger's soles.

The air pump 26 and the control valve 27 are preferably provided on a rear surface of the plate 21, that is, a rear surface of a surface of the plate 21 on which the bladder unit 22 is provided.

A control unit 28 controls the power source supply to the heat wire 24a, and controls operations of the air pump 26 and the control valve 27.

When the operation of the foot massage assembly 20 is required by a switch operation of the passenger or the like, the control unit 28 supplies a power source to the heat wire 24a while operating the air pump 26 and the control valve 27.

At the present time, the control unit 28 may also control only the foot massage function to be used by operating only the air pump 26 and the control valve 27, or only the heating function heating the feet to be used by operating only the heat wire 24a according to the passenger's demand.

Furthermore, when the foot rest 12 is operated in conjunction with the operation of the foot rest 12 provided on the seatback 11, the control unit 28 may also control the heat wire, the air pump 26, and the control valve 27 to be operated.

An operation of the foot massage assembly for the vehicle according to various exemplary embodiments of the present invention having the above configuration will be referred to as follows.

When a user occupying a rear seat operates an operation button for using the foot massage assembly 20, the seat 1 provided with the foot massage assembly 20 moves forward, and the seatback 11 is folded.

Accordingly, the foot rest 12 is deployed. If only the foot rest 12 is used without using the foot massage assembly 20, the foot rest 12 supports the passenger's soles. However, in the case of the use of the foot massage assembly 20, the foot rest 12 is configured to support the passenger's heel and calf.

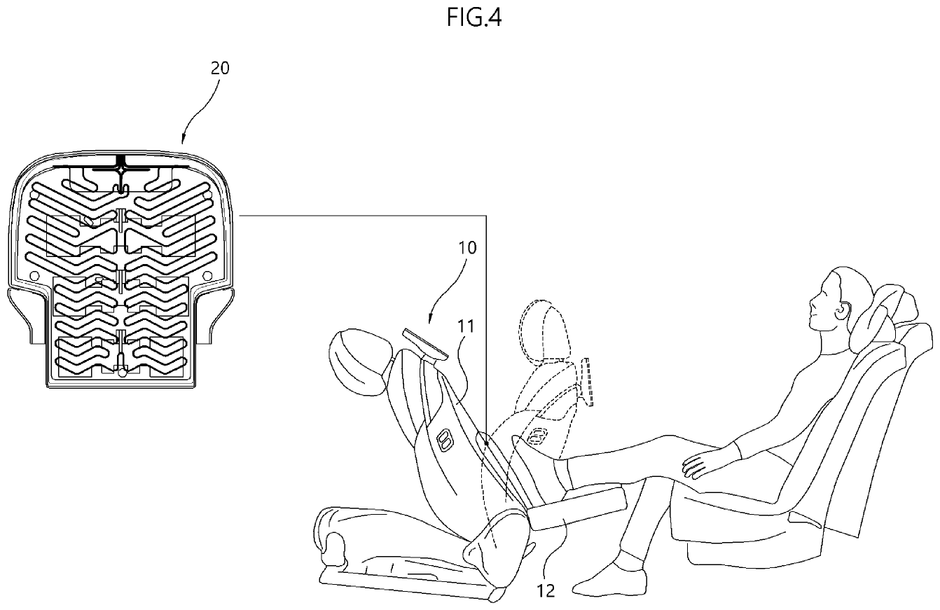

As described above, when the seatback 11 is folded and the foot rest 12 is deployed, as illustrated in FIG. 4, the passenger contacts his or her soles on the cover 25 while supporting the feet on the foot rest 12.

When the foot rest 12 is deployed, the control unit 28 operates the air pump 26 and the control valve 27 such that the bladder cell 22b repeats expansion and restoration to perform the foot massage function. Furthermore, the control unit 28 provides the heating function during foot massage by supplying the power source to the heat wire 24a.

Meanwhile, the movement of the seat, the folding of the seatback 11, and the deployment of the foot rest 12 may also be manually provided, and in a state where the deployment of the foot rest 12 is completed, the passenger may operate the switch to operate the foot massage assembly 20.

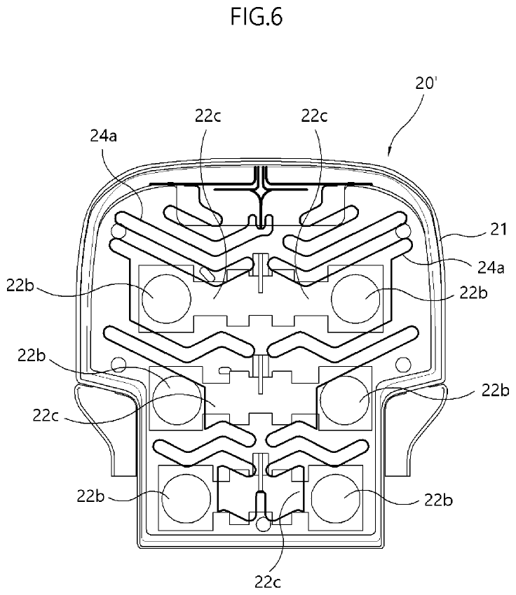

FIG. 6 illustrates a foot massage assembly 20 for a vehicle according to various exemplary embodiments of the present invention.

The exemplary embodiment of the present invention has the arrangement of the heat wire 24a on the heat wire mat 24 different from that of the aforementioned exemplary embodiment of the present invention.

As in the aforementioned exemplary embodiment of the present invention, when the heat wire 24a is disposed to overlap the bladder cell 22b, the heat wire 24a overlapping the bladder cell 22b is highlighted on the cover 25 upon expansion of the bladder cell 22b, which may become the cause of lowering the quality of the external appearance. Furthermore, as the expansion and restoration of the bladder cell 22b are repeated, the heat wire 24a may be damaged, not performing the heating function.

To prevent such a problem, when being arranged on the heat wire mat 24, the heat wires 24a are basically arranged on the entire heat wire mat 24, and may be provided in a region, in which the bladder cell 22b is not provided, not to overlap the bladder cell 22b by bypassing the bladder cell 22b. When air is supplied through the air hose 22c, the center portion of the bladder cell 22b is maximally expanded, and the heat wire 24a bypasses not to overlap the bladder cell 22b such that it is possible to prevent the phenomenon in which the heat wire 24a is highlighted upon expansion of the bladder cell 22b, or in which the heat wire 24a is damaged by the repeated operation of the bladder cell 22b.

Here, the fact that the heat wire 24a bypasses without overlapping the bladder cell 22b means that the heat wire 24a is provided not to be highlighted upon expansion of the bladder cell 22b even if the heat wire 24a substantially, partially overlaps the bladder cell 22b. Therefore, the heat wire 24a may be provided not to overlap the bladder cell 22b on the maximally expanded portion and the portion adjacent thereto in the bladder cell 22b.

As described above, as the heat wire 24a is provided in the region in which the bladder cell 22b is not provided, the heat wire 24a is not highlighted on the cover 25 even if the bladder cell 22b is expanded, improving the quality of the appearance.

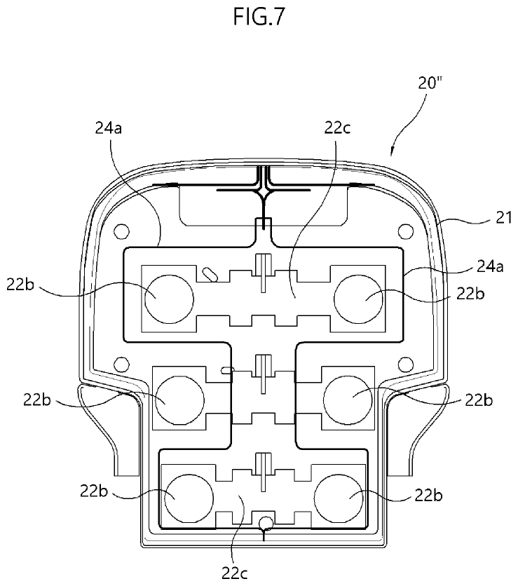

Meanwhile, FIG. 7 illustrates a foot massage assembly 20 for a vehicle according to various exemplary embodiments of the present invention.

Even in the exemplary embodiment of the present invention, only the arrangement of the heat wire 24a on the heat wire mat 24 is different and the rest of the configuration is the same as in the aforementioned exemplary embodiment of the present invention.

The exemplary embodiment of the present invention is the same as in the aforementioned exemplary embodiment in that when the heat wire 24a is provided on the heat wire mat 24, the heat wire 24a bypasses to the region in which the bladder cell 22b is not provided without overlapping the bladder cell 22b. However, the heat wire 24a may be provided to partially overlap the air hose 22c and to be bypassed without partially overlapping it.

At the present time, when the heat wire 24a bypasses to the circumferences of the bladder cell 22b and the air hose 22c with respect to any one bladder cell 22b, the heat wire 24a bypasses the bladder cell 22b with respect to other bladder cells 22b disposed above and below the bladder cell 22b and is provided to overlap the air hose 22c.

When all of the heat wires 24a do not overlap the air hose 22c either, the heating function is significantly lowered during the foot massage of the passenger such that it is possible to prevent the heating function from being significantly lowered by overlapping some heat wires with the air hose 22c.

As described above, the heat wire 24a is also disposed inside the bladder unit 22 while all of the heat wires 24a bypass the bladder cell 22b and the heat wire 24a partially bypass the air hose 22c and partially overlap the air hose 22c.

For example, as illustrated in FIG. 7, if the bladder cells 22b are arranged by three stages in the vertical direction of the felt 22a, the heat wire 24a bypasses the bladder cell 22b and the air hose 22c positioned on the upper end portion and lower end portion of the felt 22a. Furthermore, with respect to the bladder cell 22b and the hose 22c positioned on the center portion of the felt 22a, the heat wire 24a is provided to vertically pass through the air hose 22c to minimize the overlapping with the air hose 22c while bypassing the bladder cell 22b.

Therefore, even if the bladder cell 22b is maximally expanded, the heat wire 24a is not provided on the bladder cell 22b such that it is possible to prevent the phenomenon in which the heat wire 24a is highlighted on the cover 25.

Furthermore, the term related to a control device such as “controller”, “control unit”, “control device” or “control module”, etc refers to a hardware device including a memory and a processor configured to execute one or more steps interpreted as an algorithm structure. The memory stores algorithm steps, and the processor executes the algorithm steps to perform one or more processes of a method in accordance with various exemplary embodiments of the present invention. The control device according to exemplary embodiments of the present invention may be implemented through a nonvolatile memory configured to store algorithms for controlling operation of various components of a vehicle or data about software commands for executing the algorithms, and a processor configured to perform operation to be described above using the data stored in the memory. The memory and the processor may be individual chips. Alternatively, the memory and the processor may be integrated in a single chip. The processor may be implemented as one or more processors. The processor may include various logic circuits and operation circuits, may process data according to a program provided from the memory, and may generate a control signal according to the processing result.

The control device may be at least one microprocessor operated by a predetermined program which may include a series of commands for carrying out the method disclosed in the aforementioned various exemplary embodiments of the present invention.

The aforementioned invention can also be embodied as computer readable codes on a computer readable recording medium. The computer readable recording medium is any data storage device that can store data which may be thereafter read by a computer system. Examples of the computer readable recording medium include hard disk drive (HDD), solid state disk (SSD), silicon disk drive (SDD), read-only memory (ROM), random-access memory (RAM), CD-ROMs, magnetic tapes, floppy discs, optical data storage devices, etc and implementation as carrier waves (e.g., transmission over the Internet).

In various exemplary embodiments of the present invention, each operation described above may be performed by a control device, and the control device may be configured by a plurality of control devices, or an integrated single control device.

In various exemplary embodiments of the present invention, the control device may be implemented in a form of hardware or software, or may be implemented in a combination of hardware and software.

For convenience in explanation and accurate definition in the appended claims, the terms “upper”, “lower”, “inner”, “outer”, “up”, “down”, “upwards”, “downwards”, “front”, “rear”, “back”, “inside”, “outside”, “inwardly”, “outwardly”, “interior”, “exterior”, “internal”, “external”, “forwards”, and “backwards” are used to describe features of the exemplary embodiments with reference to the positions of such features as displayed in the figures. It will be further understood that the term “connect” or its derivatives refer both to direct and indirect connection.

The foregoing descriptions of specific exemplary embodiments of the present invention have been presented for purposes of illustration and description. They are not intended to be exhaustive or to limit the present invention to the precise forms disclosed, and obviously many modifications and variations are possible in light of the above teachings. The exemplary embodiments were chosen and described to explain certain principles of the present invention and their practical application, to enable others skilled in the art to make and utilize various exemplary embodiments of the present invention, as well as various alternatives and modifications thereof. It is intended that the scope of the present invention be defined by the Claims appended hereto and their equivalents.

What is claimed is:

1. A foot massage apparatus for a vehicle, the foot massage apparatus comprising:
a plate mounted on a seatback;
a bladder unit spaced from the plate at an interval and provided with a plurality of bladder cells, wherein air is supplied to or exhausted from an inside of the plurality of bladder cells for pressurizing passenger's soles; and
a heat wire mat positioned to cover the bladder unit and provided with a heat wire.

2. The foot massage apparatus of claim 1,
wherein the heat wire is uniformly distributed and provided on the heat wire mat.

3. The foot massage apparatus of claim 1,
wherein the heat wire is provided in a region in which the plurality of bladder cells is not provided.

4. The foot massage apparatus of claim 3,
wherein the heat wire is provided in a region in which an air hose for supplying pressurized air to the plurality of bladder cells is not provided.

5. The foot massage apparatus of claim 3,
wherein the heat wire is provided to overlap an air hose for supplying pressurized air to the plurality of bladder cells.

6. The foot massage apparatus of claim 1,
wherein the heat wire is arranged to be tilted with respect to a vertical direction or a horizontal direction of the heat wire mat.

7. The foot massage apparatus of claim 1,
wherein the heat wire is provided on the heat wire mat so that left and right sides of the heat wire are linearly symmetric to each other.

8. The foot massage apparatus of claim 1, further including:
an air pump configured for pressurizing the air to supply the pressurized air to the plurality of bladder cells through an air hose connected to the plurality of bladder cells; and
a control valve provided between the air pump and the air hose for selectively supplying the pressurized air to the plurality of bladder cells to selectively interrupt flow of the air.

9. The foot massage apparatus of claim 8, further including:
a control unit electrically connected to the heat wire mat, the control valve and the air pump and configured for operating the air pump to supply the pressurized air to the plurality of bladder cells and connecting a power source to the heat wire.

10. The foot massage apparatus of claim 1,
wherein the bladder unit is provided with a felt coupled to the plate, and
wherein the plurality of bladder cells is attached to the felt at an interval.

11. The foot massage apparatus of claim 10,
wherein the plurality of bladder cells is attached to the felt by thermal fusing.

12. The foot massage apparatus of claim 1,
wherein an elastic member is provided between the bladder unit and the heat wire mat for buffering therebetween.

13. The foot massage apparatus of claim 12,
wherein a through hole is formed in the elastic member in a portion thereof, in which the plurality of bladder cells is disposed.

14. The foot massage apparatus of claim 1, further including:
a cover for covering the heat wire mat.

15. The foot massage apparatus of claim 1,
wherein the foot massage apparatus is selectively covered by a foot rest hinged to the seatback so that the foot massage apparatus is exposed when the foot rest hinged to the seatback is deployed.

16. A foot massage apparatus for a vehicle, the foot massage apparatus comprising:
a plate mounted on a seatback;
a bladder unit spaced from the plate at an interval and provided with a plurality of bladder cells, wherein air is supplied to or exhausted from an inside of the plurality of bladder cells for pressurizing passenger's soles; and
a heat wire mat having a heat wire arranged in a region in which the plurality of bladder cells is not provided and positioned to cover the bladder unit.

17. The foot massage apparatus of claim 16,
wherein the heat wire is uniformly distributed and provided in a region other than the plurality of bladder cells.

18. The foot massage apparatus of claim 16,
wherein the heat wire is provided to bypass an air hose fluidically connected to a bladder cell among the plurality of bladder cells, and
wherein the heat wire is provided to overlap the air hose connected to other bladder cells disposed above and below the bladder cell among the plurality of bladder cells.

19. The foot massage apparatus of claim 18,
wherein the heat wire is arranged to overlap the air hose to be perpendicular to a direction in which the air hose is arranged upon overlapping the air hose.
